(19) United States
(12) Patent Application Publication
(10) Pub. No.: US 2024/0009381 A1
(43) Pub. Date: Jan. 11, 2024

(54) DRUG HYPERSENSITIVITY PREVENTION SYSTEM

(71) Applicants: SEOUL NATIONAL UNIVERSITY R&DB FOUNDATION, Seoul (KR); CEBIKA INC., Uiwang-si, Gyeonggi-do (KR)

(72) Inventors: Hye-Ryun KANG, Seoul (KR); Jung Chan LEE, Seoul (KR); Kyoung Jin LEE, Seongnam si (KR); Byeong Chic CHO, Seongnam-si, (KR); Sung Ho KO, Seoul (KR)

(21) Appl. No.: 18/245,888

(22) PCT Filed: Sep. 17, 2021
(86) PCT No.: PCT/KR2021/012809

§ 371 Date: Mar. 17, 2023

(30) Foreign Application Priority Data
Sep. 18, 2020 (KR) 10 2020 -0120775

(51) Int. Cl. A61M 5/142; A61M 5/172; A61M 39/22

(52) U.S. Cl. CPC A61M 5/14228; A61M 5/172; A61M 39/22

(57) ABSTRACT

Provided is a drug hypersensitivity prevention system. A drug hypersensitivity prevention system according to an embodiment of the present application may comprise: a first fluid storage unit 10; a second fluid storage unit 20; a control device 30 including a first pump 31 installed in a discharge line connected to the first fluid storage unit 10 so as to control an amount of a first fluid discharged from the first fluid storage unit 10, and a second pump 32 installed in a discharge line connected to the second fluid storage unit 20 so as to control an amount of a second fluid discharged from the second fluid storage unit 20; a mixing unit 40 in which the first fluid discharged from the first fluid storage unit 10 and the second fluid discharged from the second fluid storage unit 20 are mixed; a pouch 50 connected to the mixing unit 40; and a catheter 60 connected to the mixing unit 40.

### TECHNICAL FIELD

The present disclosure relates to a system for preventing hypersensitivity when a drug requiring dilution and stepwise weight increase, particularly, a drug with a high risk of hypersensitivity is administered.

### BACKGROUND ART

A technique of adjusting the amount of a fluid flowing through a pipe to a desired amount and discharging the fluid is used in various fields. In the medical field, drugs to be administered or injected to patients need to be administered according to a designated usage, and among these usages, an injection amount, an injection rate, and an injection time are very important factors.

Each person has a different degree of sensitivity to drugs, and some hypersensitivity is relatively prevalent, but theoretically all drugs can cause hypersensitivity, and there is typical hypersensitivity such as mast cell activation reactions, for example, urticaria, angioedema, and anaphylaxis or lymphocyte reactions such as rashes.

In the case of hypersensitivity, some drugs can be replaced with other drugs, but when there is no substitute drug or replacement of a given drug can cause significant reduction in its effect, stopping the administration of the drug that caused hypersensitivity may lead to treatment failure.

In order to prevent recurrence of drug hypersensitivity, desensitization therapy in which the injection amount of the drug is slowly increased while gradually increasing the dose from a very small amount, has been carried out. Currently, the most widely used desensitization therapy is a 3-bag (1:100 dilution, 1:10 dilution, 1:1 undiluted solution), 12-step protocol which can be applied to various drugs.

However, since this protocol has several problems in that the entire protocol is performed manually by pharmacists and nursing personnel, in the case of toxic anticancer drugs, they may be exposed to the anticancer drugs during dilution and replacement; that the speed of a pump needs to be manually adjusted to increase the dose at each stage; that due to the nature of a discharge line and the volume of a catheter, a small amount of drug may still remain therein without being injected, thus making it difficult to accurately administer the drugs at a desired concentration and volume.

In addition, in the case of desensitization therapy according to the related art, there is a limitation that the history of changes in the injection rate (speed, volume, time) of a fluid injected during implementation cannot be confirmed on a system, and thus all operations are manually recorded, which increases the burden on nursing personnel.

Korean Patent No. 10-2080347 discloses an automatic drug injection device for performing desensitization therapy and suggests a device for injecting a dilution fluid generated using a drug injection pump and a diluent injection pump, but gradually increases the injection rate of the dilution fluid of a single concentration, and the concentration cannot be changed during implementation, and there is a risk that nursing personnel may be exposed to toxic drugs during priming and when removing the catheter from an object after drug injection is finished.

(Patent Document 1) Korean Patent Laid-open Publication No. 10-2019-0059047 (May 30, 2019)

(Patent Document 2) Korea Patent No. 10-0499301 (Jun. 24, 2005)

(Patent Document 3) Japanese Patent Laid-open Publication No. 2013-525066 (Jun. 20, 2013)

### DETAILED DESCRIPTION OF THE INVENTION

### Technical Problem

The present disclosure has been devised to solve the above problems. Specifically, the present disclosure provides a system in which, when a drug with a high risk of hypersensitivity is administered, the drug is automatically diluted without the manual work of dedicated personnel and is then injected stepwise by increasing the speed, volume and concentration so that hypersensitivity can be minimized.

### Technical Solution

In order to solve the above problems, according to an aspect of the present disclosure, there is provided a drug hypersensitivity prevention system including: a first fluid storage unit 10; a second fluid storage unit 20; a control device 30 including a first pump 31 installed on a discharge line connected to the first fluid storage unit 10 so as to control an amount of a first fluid discharged from the first fluid storage unit 10, and a second pump 32 installed on a discharge line connected to the second fluid storage unit so as to control an amount of a second fluid discharged from the second fluid storage unit 20; a mixing unit 40 in which the first fluid discharged from the first fluid storage unit 10 and the second fluid discharged from the second fluid storage unit 20 are mixed; a pouch 50 connected to the mixing unit 40; and a catheter 60 connected to the mixing unit 40.

In an embodiment, a first valve 71 may be installed between the mixing unit 40 and the pouch 50, and a second valve 72 may be installed between the mixing unit 40 and the catheter 60.

In an embodiment, the control device 30 may further include: an input unit 33 to which a plurality of input variables for implementing desensitization therapy are input; and a control unit 34 configured to generate a desensitization therapy program according to a plurality of input variables as the plurality of input variables are input through the input unit 33 and to control the operations of one or more of the first pump 31, the second pump 32, the first valve 71, and the second valve 72 according to the generated desensitization therapy program.

In an embodiment, the inputted plurality of input variables may include three or more of a total number of injection operations for implementing desensitization therapy, an initial injection rate, an injection time for each injection operation, a total amount of injection, and an increase rate for each operation.

In an embodiment, the drug hypersensitivity prevention system may further include a biometric information measuring unit 80 configured to measure biometric information of a fluid injection object, wherein, when the biometric information measured by the biometric information measuring unit 80 is out of a predetermined range, the control device 30 may be configured to control the operations of the first pump 31 and the second pump 32 to be stopped.

In an embodiment, the biometric information may include one or more of electrocardiogram, blood oxygen saturation, and blood pressure.

In an embodiment, the drug hypersensitivity prevention system may further include a sensor S installed downstream of the mixing unit 40 and configured to detect a flow rate of a fluid discharged through the catheter 60.

According to another aspect of the present disclosure, there is provided a drug hypersensitivity prevention system including: a first fluid storage unit 10; a second fluid storage unit 20; a first pump 31 installed on a discharge line connected to the second fluid storage unit 20 and configured to control an amount of a second fluid discharged from the second fluid storage unit 20; a mixing unit 40 in which a first fluid discharged from the first fluid storage unit 10 and the second fluid discharged from the second fluid storage unit 20 are mixed; a second pump 32 installed downstream of the mixing unit a pouch 50 connected to the mixing unit 40; and a catheter 60 connected to the mixing unit 40.

According to another aspect of the present disclosure, there is provided a drug hypersensitivity prevention system including: a first fluid storage unit 10; a second fluid storage unit 20; a flow rate control unit 90 installed on a discharge line connected to the first fluid storage unit 10 and configured to control an amount of a first fluid discharged from the first fluid storage unit 10; a second pump 32 installed on a discharge line connected to the second fluid storage unit 10 and configured to control an amount of a second fluid discharged from the second fluid storage unit 20; a mixing unit 40 in which the first fluid discharged from the first fluid storage unit 10 and the second fluid discharged from the second fluid storage unit 20 are mixed; a pouch 50 connected to the mixing unit 40; and a catheter 60 connected to the mixing unit 40.

According to another aspect of the present disclosure, there is provided a drug hypersensitivity prevention system including: a first fluid storage unit 10; a second fluid storage unit 20; a flow rate control unit 90 installed on a discharge line connected to the first fluid storage unit 10 and configured to control an amount of a first fluid discharged from the first fluid storage unit 10; a first pump 31 and a second pump 32 configured to control an amount of a second fluid discharged from the second fluid storage unit 20 and installed in parallel to each other; a mixing unit 40 in which the first fluid discharged from the first fluid storage unit 10 and the second fluid discharged from the second fluid storage unit 20 are mixed; a pouch 50 connected to the mixing unit 40; and a catheter 60 connected to the mixing unit 40.

In an embodiment, the drug hypersensitivity prevention system may further include a backflow prevention valve 73 installed at a rear end of the flow rate control unit 90 and a front end of the mixing unit 40 and configured to limit movement of a fluid from the mixing unit to the flow rate control unit 90.

According to another aspect of the present disclosure, there is provided a controlling method of the drug hypersensitivity prevention system described above, the controlling method including: (a) opening the second valve 72 and closing the first valve 71 so that a fluid accommodated in the mixing unit 40 is discharged only to the catheter for a first predetermined amount of time; (b) controlling an operation of the first pump 31 so that a first fluid stored in the first fluid storage unit 10 is discharged to an outside through the catheter 60; and (c) controlling an operation of a second pump 32 for a second predetermined amount of time based on a length of a discharge line between the second fluid storage unit 20 and the mixing unit 40.

In an embodiment, the controlling method may further include, after (c): (d) opening the first valve 71 and closing the second valve 72 so that the fluid accommodated in the mixing unit 40 is discharged only to the pouch 50; (e) generating a desensitization therapy program according to a plurality of input variables as the plurality of input variables are inputted through the input unit 33; and (f) controlling the operations of the first pump 31 and the second pump 32 according to the generated desensitization therapy program.

In an embodiment, the controlling method may further include, after (f): (g) opening the second valve 72 and closing the first valve 71 so that the fluid accommodated in the mixing unit 40 is discharged only to the catheter 60; and (h) controlling the operations of the first pump 31 and the second pump 32 according to the generated desensitization therapy program.

In an embodiment, the controlling method may further include, after (h): (i) terminating an operation of a first pump 31 and a second pump 32 if air is detected by an air detection sensor or a total injection time according to the generated desensitization therapy program has elapsed; and (j) operating the first pump 31 for a predetermined amount of time so that a first fluid stored in the first fluid storage unit 10 is discharged through the catheter 60.

In an embodiment, in a plurality of injection operations included in the desensitization therapy program, the injection rate may increase as an injection operation proceeds.

In an embodiment, the plurality of injection operations may include a plurality of injection operations in which an injection rate increases as an injection operation proceeds but an injection concentration of a predetermined material included in the mixed fluid is the same even if the injection operation proceeds, and may further include a plurality of injection operations in which the injection concentration increases as the injection operation proceeds but the injection rate is the same even if the injection operation proceeds.

According to another aspect of the present disclosure, there is provided a program stored in a computer-readable recording medium to execute the method described above.

### Effects of the Invention

According to the present application described above, when a drug with a high risk of hypersensitivity requiring dilution and stepwise injection is injected, a program for minimizing hypersensitivity is automatically performed so that inconveniences of manual manipulation of each operation by nursing personnel are removed and thus the implementation of desensitization therapy for preventing hypersensitivity can be facilitated.

In addition, because the concentration and injection rate of the drug are the main risk factors of the occurrence of hypersensitivity, a desensitization therapy program in which the dose of a drug with a high risk of hypersensitivity is increased starting from a lower concentration and then the speed is increased and then, when a certain speed reaches, the constant speed is maintained and the concentration is stepwise increased, is generated, and control thereof is performed so that the risk of the occurrence of hypersensitivity can be minimized.

In addition, when a catheter is directly connected to a mixing unit and a desensitization therapy program including multiple injection operations is executed, a phenomenon, in which the concentration set in the previous injection operation affects subsequent operations, can be minimized.

In addition, a controlling method from a priming operation to a washing operation is fully automated so that the convenience of performing desensitization therapy can be enhanced.

In addition, a pouch capable of storing a separate fluid is provided so that, when a toxic anticancer agent is administered, direct exposure to the anticancer agent can be avoided.

In addition, when biometric information measured through a biometric information measurement unit, the state of a fluid injection object and the injected flow rate in real time through a sensor installed on a discharge line are detected and the detected result is out of a preset normal range, a warning signal is sent to the outside through an alarm unit, and a system operation is stopped so that hypersensitivity of the fluid injection object can be rapidly checked and handled.

### DESCRIPTION OF THE DRAWINGS

FIG. 1 is a perspective view of a drug hypersensitivity prevention system according to a first embodiment of the present disclosure;

FIG. 2 is a schematic block diagram of the drug hypersensitivity prevention system of FIG. 1;

FIG. 3 is a schematic block diagram of a drug hypersensitivity prevention system according to a second embodiment of the present disclosure;

FIG. 4 is a schematic block diagram of a drug hypersensitivity prevention system according to a third embodiment of the present disclosure;

FIG. 5 is a schematic block diagram of a drug hypersensitivity prevention system according to a fourth embodiment of the present disclosure;

FIG. 6 is a flowchart illustrating a controlling method of a drug hypersensitivity prevention system according to an embodiment of the present disclosure;

FIG. 7 is a flowchart illustrating a priming operation in the controlling method of FIG. 6;

FIG. 8 is a flowchart illustrating a preparation operation in the controlling method of FIG. 6;

FIG. 9 is a flowchart illustrating an injection operation in the controlling method of FIGS. 6; and

FIG. 10 is a flowchart illustrating a washing operation in the controlling method of FIG. 6.

### MODE OF THE INVENTION

Hereinafter, the present disclosure will be described in detail with reference to the accompanying drawings.

Hereinafter, it is noted that a system is “things”.

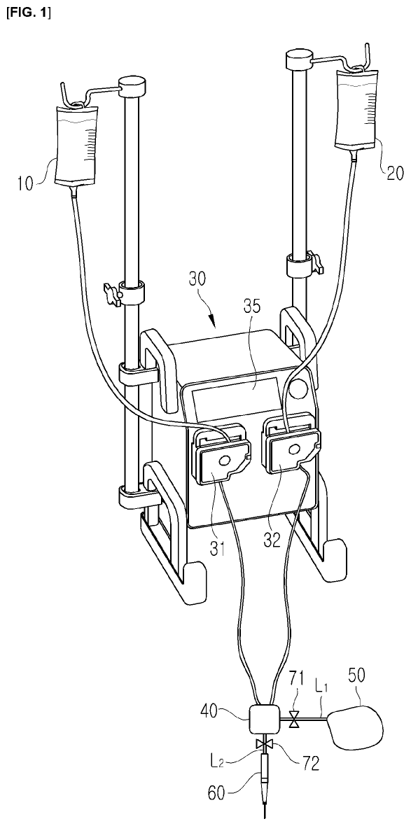

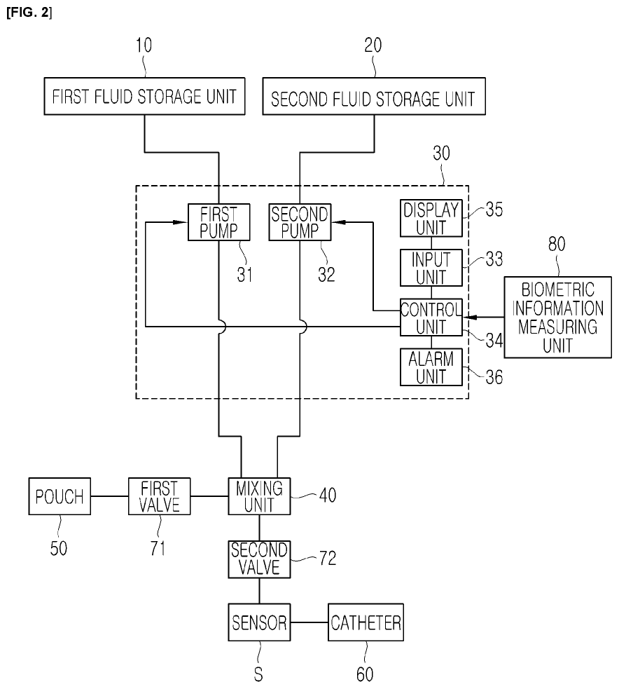

First, a drug hypersensitivity prevention system according to a first embodiment of the present disclosure will be described in more detail with reference to FIGS. 1 and 2.

The drug hypersensitivity prevention system according to the embodiment of the present disclosure is used to mix a first fluid and a second fluid and to inject the mixed fluid through a catheter 60, and may include a first fluid storage unit 10 in which the first fluid is stored, and a second fluid storage unit 20 in which the second fluid is stored.

Here, the first fluid may be a dilution fluid, for example, a physiological saline solution or glucose solution.

In addition, the second fluid may be a therapeutic drug, and may be applied to cytotoxic anti-cancer drugs, targeted anti-cancer drugs, immuno-anticancer drugs, and all other drugs with a high risk of hypersensitivity.

That is, the drug hypersensitivity prevention system according to the embodiment of the present disclosure was originally intended for injecting the second fluid alone, but injecting the second fluid, which is a therapeutic drug, as an undiluted solution without dilution has a high possibility of causing side effects such as hypersensitivity; therefore, the drug hypersensitivity prevention system of the present disclosure has been modified such that the second fluid is diluted with the first fluid so as to inject the therapeutic drug at a diluted concentration.

The first fluid storage unit 10 and the second fluid storage unit 20 may be connected to a first pump 31 and a second pump 32 that control the amount of a fluid discharged from each storage unit. The first pump 31 may be installed on a discharge line of the first fluid storage unit 10, and the second pump 32 may be installed on a discharge line of the second fluid storage unit 20. The first pump 31 and the second pump 32 may be, for example, a peristaltic pump, and the first pump 31 and the second pump 32 canpump the fluid stored in each fluid storage unit in a principle in which a discharge line is installed in the pump so that the fluid is discharged while a roller provided in the pump pressurizes the discharge line at regular intervals.

The first fluid and the second fluid pumped by the first pump 31 and the second pump 32 may be mixed by a mixing unit 40 provided downstream of the first pump 31 and the second pump 32. To this end, the mixing unit 40 is fluidly connected to the first fluid storage unit 10 and the second fluid storage unit 20, and has a predetermined space therein so that the fluids can be mixed. The fluids pumped by the pump are mixed in the mixing unit 40 to generate a mixed fluid.

Here, the resolution of the first pump 31 may be higher than the resolution of the second pump 32, wherein the resolution means the pumping speed of the fluid achieved for each rotation of the roller provided in the pump, and the lower the resolution, the more precise the flow rate control. For example, when the resolution of one pump is 0.02 ml/rev and the resolution of the other pump is 0.06 ml/rev, in the former case, the flow rate can be adjusted in units of 0.02 ml, but in the case of the latter, the flow rate can be adjusted in units of 0.06 ml only. Thus, a more precise control of the second fluid discharged from the second fluid storage unit 20 is possible.

The mixing unit 40, a first discharge line L₁ and a second discharge line L₂ are connected to each other, and a pouch 50 is connected to the end of the first discharge line L₁, and a catheter 60 is connected to the end of the second discharge line L₂.

The fluid injected into a target for fluid injection flows along the second discharge line L₂. The first fluid used during an air removal operation (priming) of a discharge line described later, and a mixed fluid having concentration set in the previous injection operation when the injection operation is changed, remain in the second fluid discharge line L₂. At this time, the longer the length of the second discharge line L₂, the higher the residual amount being increased in proportion thereto, and a large error may occur in the process of precisely controlling a desired concentration and a flow rate. Thus, the shorter the length of the second discharge line L₂, the lesser the effect of the air removal operation or the fluid used in the previous injection operation. More preferably, because the catheter 60 is directly connected to the mixing unit 40 without the second fluid discharge line L₂, the effect of the remaining fluid may also be minimized.

A first valve 71 may be installed on the first fluid discharge line L₁, and a second valve 72 may be installed on the second fluid discharge line L₂. In another embodiment, the first fluid discharge line L₁ is branched from the second fluid discharge line L₂, and a 3-way valve may be installed at the branched point.

When one of the first valve 71 and the second valve 72 is opened, the other may be closed, and thus, the fluid introduced into the mixing unit 40 may flow through the pouch 50 or the catheter 60.

The pouch 50 is in fluid communication with the first discharge line Li and stores the fluid introduced through the first discharge line L₁. As described below, the pumped fluid may be stored by operating the pump to remove air in each discharge line, and the second fluid used to remove air in the discharge line of the second fluid storage unit 20 may be stored. Thus, the risk in which the second fluid, which may have toxicity, is exposed to the outside through the catheter 60 and nursing personnel is exposed to the second fluid, is prevented.

In addition, the fluid used in the air removal operation and mixed in the mixing unit 40 is stored so that the mixed fluid can be injected with the initial concentration and flow rate of desensitization therapy. A detailed description of this will be provided later.

The catheter 60 is in fluid communication with the second discharge line L₂ and is inserted into a blood vessel of the target for fluid injection to inject a mixed fluid.

The operation of the drug hypersensitivity prevention system according to an embodiment of the present disclosure may be controlled by the control device 30.

Specifically, the control device 30 controls the concentration of the second fluid discharged through the catheter 60 and the injection rate of the mixed fluid, and the like, and may include a first pump 31 and a second pump 32, and may further include an input unit 33, a control unit 34, a display unit 35, and an alarm unit 36.

The input unit 33 is a portion through which a number of input variables are input to perform desensitization therapy, and may be provided as a separate input device, such as a keyboard, but is preferably provided in the form of a touch panel integrated with the display unit 35.

As a plurality of input variables are input through the input unit 33, the control unit 34 generates a desensitization therapy program corresponding to the input plurality of input variables and controls the operation of one or more of the first pump 31, the second pump 32, the first valve 71, and the second valve 72 according to the generated program.

Here, the plurality of input variables may include the total number of injection operations for implementing desensitization therapy, an initial injection rate, an injection time for each injection operation, a total amount of injection, and an increase rate for each operation. Each injection operation may include variables of injection rate and injection time, and for example, when there are 15 injection operations, the injection rate for each injection operation is the same as 1 ml/s and the injection time is also the same as 15 seconds, the total injection time will be 225 seconds, and the total amount of injection will be 225 ml.

Three or more input variables among the total number of injection operations, an initial injection rate, an injection time for each injection operation, a total amount of injection, and an injection rate for each injection operation are input to the input unit 33, and as the three or more input variables are input, a desensitization therapy program can be set by the operation of the control unit 34.

Because the input variables input to the input unit 33 are diverse, a plurality of desensitization therapy programs may exist.

In another embodiment of the present disclosure, drug injection patterns (injection concentration, injection rate) over time may be pre-stored in the form of graphs, respectively, and the pre-stored drug injection patterns in the form of graphs may be displayed. When a drug injection pattern to be injected is selected from among a plurality of displayed drug injection patterns, a desensitization therapy program may also be set accordingly.

The control unit 34 controls the operation of one or more of the first pump 31, the second pump 32, the first valve 71, and the second valve 72 to match each stage of the set desensitization therapy program, and accordingly, fluid injection can be performed according to the set desensitization therapy program.

In one embodiment, the desensitization therapy program may be a program in which the injection rate increases as the injection operation proceeds. When the second fluid, which is a therapeutic drug, is injected at a high injection rate from the beginning, there is a high probability of hypersensitivity; however, if the injection is performed in such a manner that it is injected at a low injection rate initially and gradually increases over time, the probability of the occurrence of hypersensitivity can be lowered.

In another embodiment, the desensitization therapy program may include a plurality of injection operations in which the injection rate increases as the injection operation proceeds but the injection concentration of a predetermined material contained in the mixed fluid of the first fluid and the second fluid is the same even if the injection operation proceeds, and a plurality of injection operations in which the injection concentration increases as the injection operation proceeds but the injection rate is the same even if the injection operation proceeds. That is, a plurality of injection operations corresponding to a fixed concentration section (injection operations in which the injection rate increases as the injection operation proceeds but the injection concentration of the predetermined material is the same), a plurality of injection operations corresponding to a fixed rate section (injection operations in which the injection rate is the same as the injection operation proceeds and the injection concentration of the predetermined material increases), and by controlling only one variable between the concentration and the speed, the load applied to each component as two variables are simultaneously controlled can be minimized.

The drug hypersensitivity prevention system according to an embodiment of the present disclosure may further include a biometric information measuring unit 80 that measures biometric information of a target for fluid injection.

The biometric information measuring unit 80 measures any one or more of the pulse, electrocardiogram, blood oxygen saturation, and blood pressure of the fluid injection object, and to this end, the biometric information measuring unit 80 may include a pulse sensor, an electrocardiogram sensor, a pulse oximeter, which is an oxygen saturation measuring device, and a blood pressure sensor.

A normal range is preset and stored for each biometric information in the control unit 34. The biometric information measured by the biometric information measuring unit 80 is transmitted to the control unit 34, and the control unit 34 compares the transmitted biometric information with the normal range preset and stored for each transmitted biometric information, and when the biometric information is out of range, the control unit 34 controls the operations of the first pump 31 and the second pump 32 to be stopped. That is, when the measured biometric information is out of the normal range, it can be considered that hypersensitivity has occurred, and in this case, the operation of the pump is stopped so that the fluid is not introduced into the target for fluid injection any more and hypersensitivity is not intensified, and through the alarm unit 36, a warning signal is notified to the outside by way of a sound, vibration, light emission, or the like.

The drug hypersensitivity prevention system according to the first embodiment may further include a sensor S for detecting the flow rate of the fluid passing through the installed discharge line. The installation position of the sensor S may vary, and the drug hypersensitivity prevention system according to the first embodiment is installed at the rear end of the mixing unit 40 to detect the flow rate of the fluid passing through the same. The flow rate detected by the sensor S corresponds to the flow rate actually injected into the target for fluid injection. Thus, a plurality of input variables are inputted to the input unit 33 and the injection rate included in each injection operation of the set desensitization therapy program is compared with the injection rate of the fluid detected by the sensor S, and when a difference of more than the preset value occurs, a warning signal is notified to the outside through the alarm unit 36.

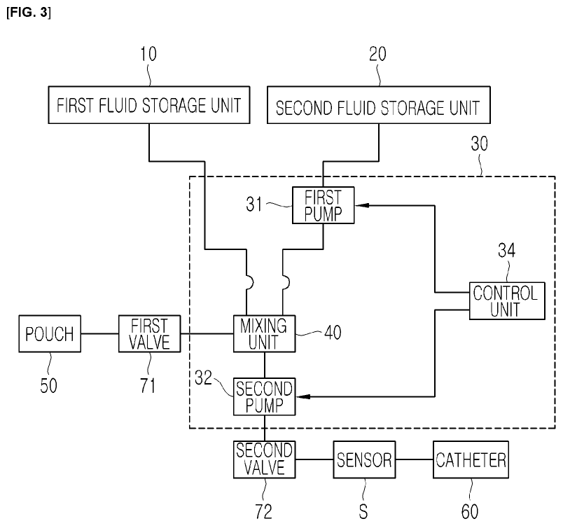

Next, drug hypersensitivity prevention systems according to second through fourth embodiments of the present disclosure will be described in more detail with reference to FIGS. 3 through 5.

Because it is the same in that the first fluid and the second fluid are mixed, the mixed fluid is injected through the catheter 60 and each pump 31 and 32 and a flow path conversion valve 70 are controlled to prevent hypersensitivity, the description of the same configuration is omitted and the difference thereof is mainly described.

First, referring to FIG. 3, the drug hypersensitivity prevention system according to the second embodiment will be described.

The drug hypersensitivity prevention system according to the first embodiment in which the first pump 31 pumps the first fluid stored in the first fluid storage unit 10, and the second pump 32 pumps the second fluid stored in the second fluid storage unit 2, and the pumped fluid is mixed in the mixing unit 40, is different from the drug hypersensitivity prevention system according to the second embodiment in which the first pump 31 is provided between the second fluid storage unit 20 and the mixing unit and the second pump 32 is provided at the rear end of the mixing unit 40 and the sensor S is installed at the rear end of the second pump 32.

That is, the second fluid stored in the second fluid storage unit 20 may be pumped by pumping of the first pump 31, and the first fluid stored in the first fluid storage unit 10 by pumping of the second pump 32 may be pumped. Here, the discharge rate of the first fluid pumped by the first fluid storage unit 10 may be the pumping rate of the second pump 32—the pumping rate of the first pump 31, and the sensor S detects the flow rate of the mixed fluid injected into the target for fluid injection and transmits a warning signal to the outside through the alarm unit 36 when the mixed fluid flows at a lower or higher rate than a predetermined rate.

As the second pump 32 directly pumps the mixed fluid mixed in the mixing unit the drug hypersensitivity prevention system according to the second embodiment has an advantage in which the injection rate of the mixed fluid to be actually injected can be precisely controlled.

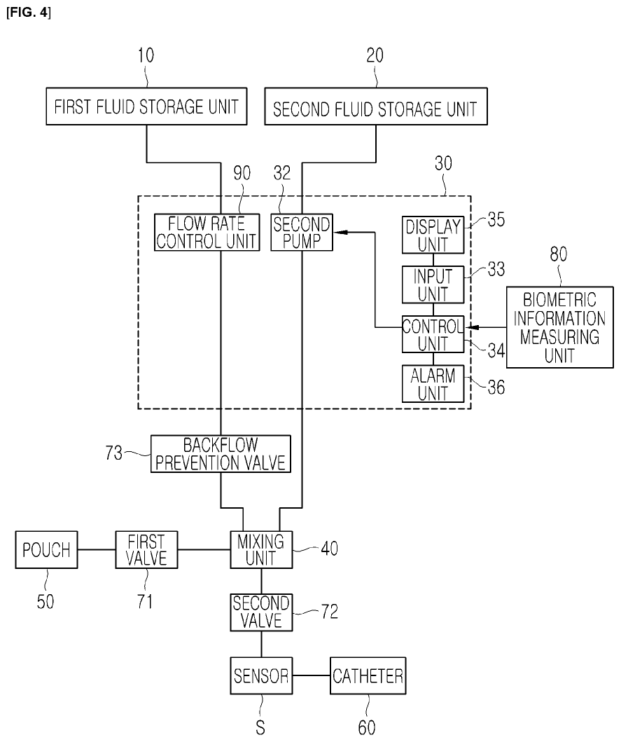

Next, a drug hypersensitivity prevention system according to a third embodiment will be described with reference to FIG. 4.

The third embodiment is different from the first embodiment in that the flow rate control unit 90 is provided instead of the first pump 31 that automatically pumps the first fluid according to the driving of a motor, and here, the flow rate control unit 90 includes a body that is generally installed in an infusion line and a roller that is vertically slidable along the infusion line with respect to the body and controls the flow rate of the first fluid discharged from the first fluid storage unit 10 according to the movement of the roller, and in another embodiment, a manipulation unit configured to be rotated like a dial is provided to adjust the flow rate with one operation by matching the flow rate to the scale written in numbers, and when the manipulation unit is aligned with the scale of the numerical value engraved with the flow rate value of the infusion to be injected, the manipulation unit may have a structure for adjusting the cross-sectional area of the fluid passage so that the fluid is injected at a flow rate corresponding to the value.

This is because the precise flow rate control of the second fluid corresponding to the anticancer agent has a greater effect on hypersensitivity than the precise flow rate control of the first fluid corresponding to the dilution fluid, even if the flow rate control unit 90, which is less precise than the first pump 31 in charge of pumping, is installed, hypersensitivity can be minimized through precise flow rate control of the second fluid by the second pump 32.

Compared to the first embodiment, the number of pumps is reduced in this embodiment so that a cost-effective effect can be attained.

In addition, the drug hypersensitivity prevention system according to the third embodiment further includes a backflow prevention valve 73 between the flow rate control unit 90 and the mixing unit 40, and thus it is possible to prevent the fluid mixed in the mixing unit 40 from backflowing into the flow rate control unit 40. In an example, a check valve may be applied as the backflow prevention valve 73.

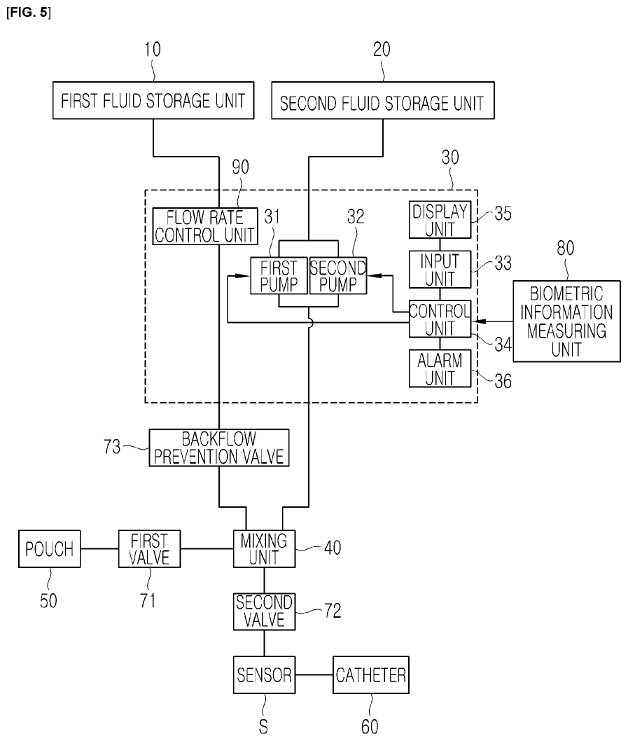

Next, a drug hypersensitivity prevention system according to a fourth embodiment will be described with reference to FIG. 5. The fourth embodiment is the same as the third embodiment in that the first fluid discharged from the first fluid storage unit 10 is controlled by the flow rate control unit 90, but the fourth embodiment is different from the third embodiment in that the second fluid is pumped from the second fluid storage unit 20 and the first pump 31 and the second pump 32 are used simultaneously.

Here, the resolution of the first pump 31 may be higher than the resolution of the second pump 32, wherein the resolution means the pumping speed of the fluid achieved for each rotation of the roller provided in the pump, and the lower the resolution, the more precise the flow rate control. In an example, when the resolution of one pump is 0.02 ml/rev and the resolution of the other pump is 0.06 ml/rev, in the former case, the flow rate can be controlled in units of 0.02 ml, but in the latter case, the flow rate can be controlled in units of 0.06 ml only.

In the drug hypersensitivity prevention system according to the fourth embodiment, the second pump 32 with low resolution is used in a low flow rate injection operation, and the first pump 31 with high resolution is used in a high flow rate injection operation, so that the flow rate control of the second fluid can be achieved more precisely.

Also, the drug hypersensitivity prevention system according to the fourth embodiment, a backflow prevention valve 73 is further provided between the flow rate control unit 90 and the mixing unit 40, so that the fluid mixed in the mixing unit 40 can be prevented from backflowing into the flow rate control unit 40. In an example, a check valve may be applied as the backflow prevention valve 73.

Hereinafter, a controlling method according to an embodiment of the present disclosure will be described in more detail by taking the drug hypersensitivity prevention system according to the first embodiment as an example.

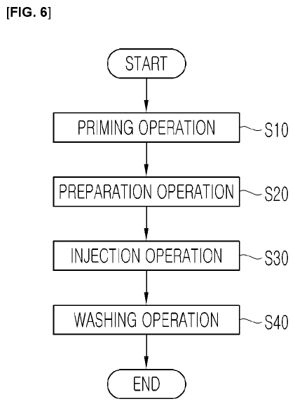

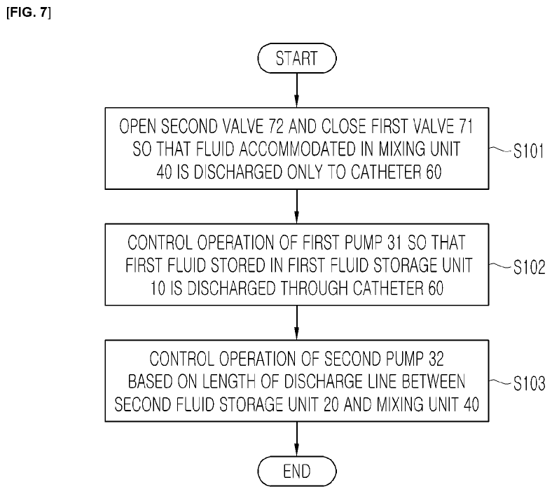

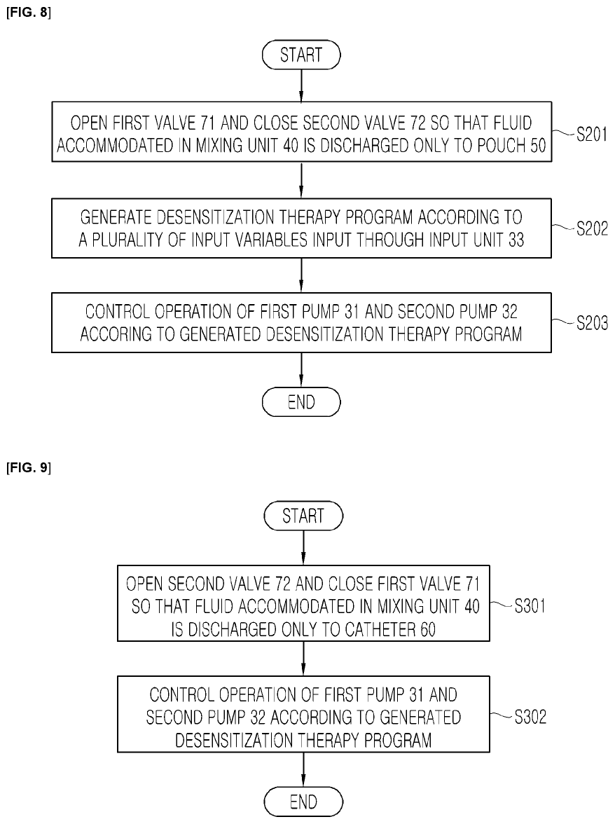

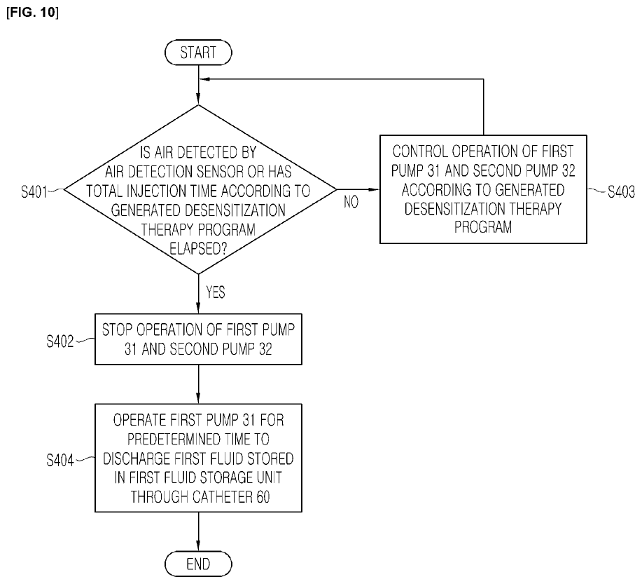

The drug hypersensitivity prevention system according to the first embodiment can be largely controlled in the order of a priming operation, a preparation operation, an injection operation, and a washing operation (FIG. 6).

The priming operation S10 is an operation for removing air in the discharge line connected to the first fluid storage unit 10 and the second fluid storage unit 20. When air is injected into the target for fluid injection, problems such as blood coagulation may occur, and thus air in the discharge line needs to be removed.

First, the second valve 72 is opened so that the fluid accommodated in the mixing unit 40 for a first predetermined amount of time is discharged only to the second discharge line L₂, and the operations of the first valve 71 and the second valve 72 are controlled so that the first valve 71 is closed (S101).

Next, the operation of the first pump 31 is controlled so that the first fluid stored in the first fluid storage unit 10 is discharged to the mixing unit 40 (S102). Thus, the first fluid, which is the dilution fluid, is discharged to the outside through the second discharge line L₂ and the catheter 60, and the air in the discharge line from the first fluid storage unit 10 to the catheter 60 is removed.

Next, the first valve 71 is opened so that the fluid accommodated in the mixing unit 40 is discharged only to the first discharge line L₁ for a second predetermined amount of time, and the operations of the first valve 71 and the second valve 72 are controlled so that the second valve 72 is closed.

Next, the operation of the second pump 32 is controlled so that the second fluid stored in the second fluid storage unit 20 is discharged to the mixing unit 40 (5103). Thus, air in the discharge line from the second fluid storage unit 20 to the mixing unit 40 is removed.

More preferably, the operation of the second pump 32 may be controlled in proportion to the length of the discharge line from the second fluid storage unit 20 to the mixing unit 40, and the operation of the second pump 32 may be controlled according to the timing when the second fluid stored in the second fluid storage unit 20 is injected into the mixing unit 40. Thus, all of the air in the discharge line from the first fluid storage unit 10 to the mixing unit 40, the air in the discharge line from the second fluid storage unit 20 to the mixing unit 40, and the air in the discharge line from the mixing unit 40 to the catheter 60 may be removed.

Next, the operations of the first pump 31 and the second pump 32 are stopped.

Once the priming operation is completed, the catheter 60 can be inserted into the target for fluid injection.

The preparation operation S20 is an operation in which, after the catheter 60 is inserted, the mixed fluid is set to be injected at an accurate concentration and flow rate before being injected into the target for fluid injection.

First, the first valve 71 is opened and the second valve 72 is closed so that the fluid contained in the mixing unit 40 is discharged only to the pouch 50 (S201).

Next, three or more input variables among the total number of injection operations, an injection rate for each operation, an injection time for each injection operation, a total amount of injection, and an increase rate for each operation are inputted to the input unit 33 to implement the desensitization therapy program (S202). Here, the input variables inputted to the input unit 33 may be determined according to the type of the second fluid stored in the second fluid storage unit 20, the amount of the second fluid, and the biometric information of the target for fluid injection measured by the biometric information measuring unit 80.

Next, the control unit 34 generates a desensitization therapy program corresponding to a plurality of input variables. In the desensitization therapy program generated by the control unit 34, injection rates of the first fluid and the second fluid (i.e., the injection rate of the mixed fluid), injection time, and injection amount are all set for each operation.

Next, the first valve 71 is opened so that the fluid accommodated in the mixing unit 40 for a predetermined amount of time is discharged only to the first discharge line L₁, and the operations of the first valve 71 and the second valve 72 are controlled so that the second valve 72 is closed.

Next, the pumping speed of the first pump 31 and the second pump 32 is controlled according to the concentration and speed of the first operation of the desensitization therapy program (S203). Thus, in the priming operation, the first fluid and the second fluid mixed in the mixing unit 40 flow into the pouch 50. In general, the desensitization therapy program starts with a very low concentration at the beginning and gradually increases the concentration, and because the concentration of the mixed fluid mixed in the mixing unit 40 in the priming operation is at a high level, when the fluid is directly injected into the target for fluid injection without this process, a shock may occur. Thus, the fluid accommodated in the mixing unit 40 may flow into the pouch 50 for a predetermined amount of time so as to be injected at a concentration set from the beginning.

The injection operation S30 is an operation in which the mixed fluid is injected into the target for fluid injection according to each injection operation of the generated desensitization therapy program, and the operations of the first pump 31 and the second pump 32 are controlled to follow the concentration set in each injection operation and injection rate.

First, the second valve 72 is opened, and the first valve 71 is closed so that the fluid accommodated in the mixing unit 40 is discharged only to the catheter 60 (S301).

Next, the desensitization therapy is performed by controlling the operations of the first pump 31 and the second pump 32 according to the generated desensitization therapy (S302).

In the injection operation, the control unit 34 determines whether the biometric information measured by the biometric information measuring unit 80 or the flow rate detected by the sensor S is within a normal range. When the biometric information or the flow rate is out of the normal range, the alarm unit 36 outputs a warning signal, and the control unit 34 stops the operations of the first pump 31 and the second pump 32.

When both biometric information and flow rate are within the normal range, the control unit 34 determines whether the total injection time according to the preset desensitization therapy program has elapsed (S401). If the total injection time according to the preset desensitization therapy program has not elapsed, the desensitization therapy program continues to be performed (S403), and if the total injection time according to the preset desensitization therapy program has elapsed, the operations of the first pump 31 and the second pump 32 are stopped (S402). Thus, the injection operation according to an embodiment of the present disclosure may be completed.

In another embodiment , when the air detection sensor provided at the rear end of the first pump 31 and the second pump 32 detects air in the discharge line, it is determined that all of the fluid stored in the first fluid storage unit 10 or the second fluid storage unit 20 is exhausted, and the operations of the first pump 31 and the second pump 32 may be stopped.

In another embodiment, when whether the total injection time according to the desensitization therapy program has elapsed and whether the air is detected by the air detection sensor are satisfied at the same time, it is determined that the injection operation is completed, and the operations of the first pump 31 and the second pump 32 may be stopped.

The washing operation S40 is an operation performed before the catheter 60 is separated from the target for fluid injection after the injection operation is terminated.

Some of the second fluid may remain in the discharge line to the catheter 60. Because the second fluid may be toxic, there may be a risk of exposure of the toxic drug to nursing personnel in the process of separating the catheter 60 from the target for fluid injection.

Thus, in the present invention, when the injection operation is terminated, the operation of the second pump 32 is stopped, and only the first pump 31 is operated to control the discharge of the first fluid to the catheter 60 (S404). Thus, the second fluid that remains in the discharge line from the fluid storage unit to the catheter 60 may be injected, and then the operation of the first pump 31 is sequentially stopped so that separation of the catheter 60 may be performed in a state in which there is no toxic drug in the second discharge line L₂.

According to the present application described above, when a drug with a high risk of hypersensitivity requiring dilution and stepwise injection is injected, a program for minimizing hypersensitivity is automatically performed, so that inconvenience of manual manipulation of each operation by nursing personnel is removed and thus the implementation of desensitization therapy for preventing hypersensitivity is facilitated.

In addition, because the concentration and injection rate of the drug are main risk factors of the occurrence of hypersensitivity, a desensitization therapy program in which the dose of a drug with a high risk of hypersensitive reactions is increased starting from a lower concentration and then the speed is increased and then, when a certain speed reaches, the constant speed is maintained and the concentration is stepwise increased, is generated, and control thereof is performed so that the risk of the occurrence of hypersensitivity can be minimized.

In addition, when a catheter is directly connected to a mixing unit and the desensitization therapy program including multiple injection operations is executed, a phenomenon in which the concentration set in the previous injection operation affects subsequent operations, can be minimized.

In addition, a controlling method from a priming operation to a washing operation is fully automated so that the convenience of performing desensitization therapy can be enhanced.

In addition, a pouch capable of storing a separate fluid is provided so that, when a toxic anticancer agent is administered, direct exposure to the anticancer agent can be avoided.

In addition, when biometric information measured through a biometric information measurement unit, the state of a target for fluid injection and the injected flow rate in real time through a sensor installed on a discharge line are detected and the detected result is out of a preset normal range, a warning signal is sent to the outside through an alarm unit, and a system operation is stopped so that hypersensitivity of the target for fluid injection can be rapidly checked and handled.

A controlling method according to an embodiment of the present invention described above may be implemented in the form of program instructions that can be executed through various computer means and recorded in a computer readable medium. The computer readable medium may include program instructions, data files, data structures, etc. alone or in combination. Program instructions recorded on the medium may be those specially designed and configured for the present invention or those known and usable to those skilled in computer software. Examples of computer-readable recording media include magnetic media such as hard disks, floppy disks and magnetic tapes, optical media such as CD-ROMs and DVDs, magneto-optical media such as floptical disks, and hardware devices specially configured to store and execute program instructions such as ROM, RAM, flash memory, and the like. Examples of program instructions include high-level language codes that can be executed by a computer using an interpreter, as well as machine language codes such as those produced by a compiler. The hardware device described above may be configured to operate as one or more software modules to perform the operations of the present invention, and vice versa.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims.

### (Explanation of Reference Numerals)

10: first fluid storage unit
20: second fluid storage unit
30: control device
31: first pump
32: second pump
33: input unit
34: control unit
36: display unit
36: alarm unit
40: mixing unit
50: pouch
60: catheter
71: first valve
72: second valve
73: backflow prevention valve
80: biometric information measuring unit
90: flow rate control unit
S: sensor
L₁: first discharge line
L₂: second discharge line

1. A drug hypersensitivity prevention system comprising:
a first fluid storage unit 10;
a second fluid storage unit 20;
a control device 30 including a first pump 31 installed on a discharge line connected to the first fluid storage unit 10 so as to control an amount of a first fluid discharged from the first fluid storage unit 10, and a second pump 32 installed on a discharge line connected to the second fluid storage unit 20 so as to control an amount of a second fluid discharged from the second fluid storage unit 20;
a mixing unit 40 in which the first fluid discharged from the first fluid storage unit 10 and the second fluid discharged from the second fluid storage unit 20 are mixed;
a pouch 50 connected to the mixing unit 40;
a catheter 60 connected to the mixing unit 40; and
a control device 30 comprising an input unit 33 to which a plurality of input variables for implementing desensitization therapy are input, and a control unit 34 configured to generate a desensitization therapy program according to a plurality of input variables as the plurality of input variables are input through the input unit 33 and to control an operation of the first pump 31 and the second pump 32 according to the generated desensitization therapy program.

2. The drug hypersensitivity prevention system of claim 1, wherein a first valve 71 is installed between the mixing unit 40 and the pouch 50, and a second valve 72 is installed between the mixing unit 40 and the catheter 60.

3. The drug hypersensitivity prevention system of claim 2, wherein:
the control unit 34 is configured to further control the operations of the first valve 71 and the second valve 72 according to the generated desensitization therapy program.

4. The drug hypersensitivity prevention system of claim 1, wherein
the inputted plurality of input variables comprise three or more of a total number of injection operations for implementing desensitization therapy, an initial injection rate, an injection time for each injection operation, a total injection amount, and an increase rate for each operation.

5. The drug hypersensitivity prevention system of claim 1, further comprising a biometric information measuring unit 80 configured to measure biometric information of a fluid injection object, wherein, when the biometric information measured by the biometric information measuring unit 80 is out of a predetermined range, the control device 30 is configured to control the operations of the first pump 31 and the second pump 32 to be stopped.

6. The drug hypersensitivity prevention system of claim 5, wherein the biometric information comprises one or more of electrocardiogram, blood oxygen saturation, and blood pressure.

7. The drug hypersensitivity prevention system of claim 5, further comprising a sensor S installed downstream of the mixing unit 40 and configured to detect a flow rate of a fluid discharged through the catheter 60.

8. A drug hypersensitivity prevention system comprising:
a first fluid storage unit 10;
a second fluid storage unit 20;
a first pump 31 installed in a discharge line connected to the second fluid storage unit 10 and configured to control an amount of a second fluid discharged from the second fluid storage unit 20;
a mixing unit 40 in which a first fluid discharged from the first fluid storage unit 10 and the second fluid discharged from the second fluid storage unit 20 are mixed;
a second pump 32 installed downstream of the mixing unit 40;
a pouch 50 connected to the mixing unit 40;
a catheter 60 connected to the mixing unit 40; and
a control device 30 comprising an input unit 33 to which a plurality of input variables for implementing desensitization therapy are inputted, and a control unit 34 configured to generate a desensitization therapy program according to a plurality of input variables as the plurality of input variables are inputted through the input unit 33 and to control the operations of the first pump 31 and the second pump 32 according to the generated desensitization therapy program.

9-11. (canceled)

12. A drug hypersensitivity prevention method as a controlling method of the drug hypersensitivity prevention system of claim 1 of claims 2 through 7, the drug hypersensitivity prevention method comprising:
(a) inputting input variables including three or more of a total number of injection operations for implementing desensitization therapy, an initial injection rate, an injection time for each injection operation, a total injection amount, and an increase rate for each operation to the input unit 33;
(b) generating a desensitization therapy program according to the input variables input in (a) by using the control unit 34; and
(c) controlling an operation of the first pump 31 and the second pump 32 according to the desensitization therapy program generated in (b) by using the control unit 34.

13-16. (canceled)

17. A program stored in a computer-readable recording medium to execute the drug hypersensitivity prevention method of claim 12.

18. The drug hypersensitivity prevention system of claim 1, wherein each injection operation of the desensitization therapy program comprises variables including an injection rate of the first fluid, an injection rate of the second fluid, and an injection time, and the desensitization therapy program generated by the control unit 34 is a program in which as the injection operation proceeds, an injection rate of a mixed fluid of the first fluid and the second fluid increases.

19. The drug hypersensitivity prevention system of claim 1, wherein each injection operation of the desensitization therapy program comprises variables including an injection rate of the first fluid, an injection rate of the second fluid, and an injection time, and the desensitization therapy program generated by the control unit 34 comprises a fixed concentration section including a plurality of same injection operations in which the injection rate of the mixed fluid of the first fluid and the second fluid increases as the injection operation proceeds but the injection concentration of the second fluid included in the mixed fluid is the same even if the injection operation proceeds, and a fixed rate section including a plurality of injection operations in which the injection rate of the mixed fluid is the same as the injection operation proceeds but the concentration of the second fluid included in the mixed fluid increases.

20. The drug hypersensitivity prevention system of claim 19, wherein the fixed rate section is a section after the fixed concentration section.

21. The drug hypersensitivity prevention method of claim 12, wherein each injection operation of the desensitization therapy program comprises variables including an injection rate of the first fluid, an injection rate of the second fluid, and an injection time, and the desensitization therapy program generated by the control unit 34 is a program in which as the injection operation proceeds, an injection rate of a mixed fluid of the first fluid and the second fluid increases.

22. The drug hypersensitivity prevention method of claim 12, wherein each injection operation of the desensitization therapy program comprises variables including an injection rate of the first fluid, an injection rate of the second fluid, and an injection time, and the desensitization therapy program generated by the control unit 34 comprises a fixed concentration section including a plurality of same injection operations in which the injection rate of the mixed fluid of the first fluid and the second fluid increases as the injection operation proceeds but the injection concentration of the second fluid included in the mixed fluid is same even if the injection operation proceeds, and a fixed rate section including a plurality of injection operations in which the injection rate of the mixed fluid is same as the injection operation proceeds but the concentration of the second fluid included in the mixed fluid increases.

23. The drug hypersensitivity prevention method of claim 22, wherein the fixed rate section is a section after the fixed concentration section.

24. The drug hypersensitivity prevention method of claim 12, further comprising:
opening the second valve 72 formed between the mixing unit 40 and the catheter 60 and closing the first valve 71 formed between the mixing unit 40 and the pouch 50 so that the fluid accommodated in the mixing unit 40 is discharged only to the catheter 60 for a first predetermined amount of time by using the control unit 34;
controlling an operation of the first pump 31 so that the first fluid stored in the first fluid storage unit 10 is discharged to an outside through the catheter 60 by using the control unit 34; and
controlling an operation of the second pump 32 for a second predetermined amount of time based on a length of a discharge line between the second fluid storage unit 20 and the mixing unit 40 by using the control unit 34.

25. The drug hypersensitivity prevention method of claim 24, further comprising opening the first valve 71 and closing the second valve 72 so that the fluid accommodated in the mixing unit 40 is discharged only to the pouch 50 by using the control unit 34.

26. The drug hypersensitivity prevention method of claim 25, further comprising:
terminating the operations of the first pump 31 and the second pump 32 by using the control unit 34 when air is detected by an air detection sensor or a total injection time according to the generated desensitization therapy program has elapsed; and
operating the first pump 31 for a predetermined amount of time so that the first fluid stored in the first fluid storage unit 10 is discharged through the catheter 60.
